(19) United States
(12) Patent Application Publication
(10) Pub. No.: US 2002/0151068 A1
(43) Pub. Date: Oct. 17, 2002

(54) Compositions and methods for the diagnosis and treatment of organophosphate toxicity

(75) Inventors: Robert Haley, Dallas, TX (US); Alan Varley, Plano, TX (US); Robert Munford, Dallas, TX (US)

Correspondence Address: Steven L. Highlander, FULBRIGHT & JAWORSKI L.L.P., Suite 2400, 600 Congress Avenue, Austin, TX, 78701, US

(73) Assignee: Board of Regents, The University of Texas System

(21) Appl. No.: 10/039,171

(22) Filed: Jan. 3, 2002

Related U.S. Application Data
(60) Provisional application No. 60/259,628, filed on Jan. 3, 2001.

(51) Int. Cl. A61K 48/00; C12N 15/86; C12N 15/861; C12N 15/867

(52) U.S. Cl. 435/456; 514/44; 435/320.1

(57) ABSTRACT

The present invention provides methods of gene therapy to prevent or treat exposure to organophosphate (OP) toxins, such as that observed in Gulf War Syndrome patients. In particular, vectors comprising the PON1 gene, which express the enzyme paraoxonase, can be used to prevent damage from OP toxins when given prior to exposure, or to reduce the toxic effects after exposure. Depending on the PON1 isotype (R or Q), protection against particular toxins may be achieved.

This application claims the priority of U.S. Provisional Application No. 60/259,628, filed Jan. 3, 2001, the disclosure of which is specifically incorporated herein by reference in its entirety.

### BACKGROUND OF THE INVENTION

I. Field of the Invention

The present invention relates to fields of toxicology, pathology and cell biology generally, and more specifically, to the use of paraoxonase 1 (PON1) genes to protect cells from toxins.

II. Brief Description of the Prior Art

Paraoxonase is a serum enzyme that hydrolyzes organophosphate compounds, aromatic carboxylic acid esters and carbamates. Esterases, such as paraoxonase, can be classified as “A” esterases or “B” esterases depending on how they interact with organophosphates (OPs). “A” esterases such as paraoxonase, other aryldialkylphophatases, and diisopropylfluorophosphatases hydrolyze OPs. “B” esterases, which are inhibited by OPs, include carboxylesterases and cholinesterases (Aldrige, 1953). OPs, which inhibit acetylcholinesterase, are very toxic. Paraoxonases can hydrolyze OPs and protect against such toxicity.

Paraoxonase can act on several substrates. One of these, the organophosphate paraoxon, is produced from parathion by cytochrome P₄₅₀ in the liver. Paraoxonase converts paraoxon to p-nitrophenol and diethylphosphate. Paraoxonase also hydrolyzes phenylacetate, a carboxylic acid ester, and thus can be considered an arylesterase. Some other common organophosphate substrates of paroxonase include diazinon, chlorpyrifos, chlorpyrifos-oxon, sarin and soman (La Du, 1992).

OPs are widely used in agriculture as insecticides and are also manufactured as chemical warfare nerve agents. They are usually applied as nontoxic sulfur (thion) derivatives. In vivo, cytochrome P₄₅₀-dependent microsomal monooxygenases perform oxidative desulfuration and activate the OPs to highly toxic oxygen (oxon) analogues. This is thought to occur in the liver, where many enzymes capable of detoxifying thions and oxons reside (La Du, 1992). If a toxic oxon escapes this method of detoxification and enters the bloodstream, serum paraoxonase can hydrolyze it before it reaches the nervous system and inhibits acetycholinesterase. Studies have shown that serum paraoxonase plays a significant role in protecting mammals from OP toxicity (Li et al., 1995; Costa et al., 1990).

Different species display different susceptibilities to OP intoxication. Most insects lack paraoxonases, which explains why OPs make good insecticides. Birds are also very susceptible to OP poisoning due to the absence of serum paraoxonase (La Du, 1992). Paraoxonase activity is present in the serum of most mammals as well as in tissues such as the liver, kidney, and small intestine. Humans have a mid-range level of paraoxonase activity compared to other mammals. Rabbit has the highest amount, followed by ferret, sheep, rat, guinea pig, goat, human, horse, and mouse (Aldridge, 1953). Paraoxonase activity in mammals is important for the detoxification of ops.

PON1 is a Ca²⁺ dependent 45-kDa glycoprotein that associates with high density lipoprotein (HDL) (La Du, 1992; Gan et al., 1991; Mackness et al., 1985). PON1 has two genetic polymorphisms which give rise to amino acid substitutions at positions 55 and 192 (Adkins et al., 1993; Humbert et al., 1993). The substitution at position 192 has been shown to determine PON1 activity towards certain substrates. The isoenzyme with an arginine at this position displays high activity towards parathion, but low activity towards sarin, diazinon and soman. The isoenzyme with a glutamine at this position displays low activity towards parathion and high activity towards sarin, diazinon and soman. Both isoforms have equal arylesterase hydrolytic activity against substrates such as phenyleacetate. This polymorphism allows individuals to be phenotyped for PON1 (Adkins et al, 1993; Humbert et al., 1993; Davies et al., 1996).

Present treatment for organophosphate poisoning consists of post-exposure administration of a combination of drugs such as carbamates, antimuscarnics, reactivators and anticonvulsants (Gray, 1984). Doctor et al. (1991) have investigated using enzymes as pretreatment drugs to prevent OP toxicity and found that exogenously administered purified cholinesterases will act as scavengers that sequester OPs before they reach their physiological targets. However, similar studies with PON1 have not been attempted, nor has anyone used “gene therapy” to address this problem.

### SUMMARY OF THE INVENTION

Thus, in accordance with the present invention, there is provided a method of protecting a cell from a toxin comprising (a) providing an expression cassette comprising a promoter active in said host cell and a gene encoding PON1 under the control of said promoter; and (b) transferring said expression cassette into said cell under conditions permitting expression of PON1. The PON1 may be type Q or type R, and the cell may express PON1 type Q or PON1 type R. The toxin may be an organophosphate, such as an organophosphate pesticide. Alternatively, the toxin may be a nerve agent. The expression cassette may further comprise a polyadenylation signal. The expression cassette also may be further comprised within a vector, for example a viral vector, such as a herpesviral vector, a retroviral vector, an adenoviral vector, an adeno-associated viral vector, a polyoma viral vector, or a vaccinia viral vector. The promoter may be a constitutive promoter, an inducible promoter or a tissue specific promoter. The expression cassette may increase PON1 type Q or type R expression by about 10-fold. The cell may be a liver cell. The said cell may express low levels of PON1 type Q or R as compared to the general population.

In another embodiment, there is provided a method of protecting a subject from a toxin comprising (a) providing an expression cassette comprising (i) a promoter active in host cells of said subject, (ii) a gene encoding PON1 under the control of said promoter; and (b) administering to said subject said expression cassette under conditions permitting expression of PON1. The toxin may be an organophosphate, such as an organophosphate pesticide. The toxin may also be a nerve agent. The administering may comprise intravenous or intraarterial administration.

In yet another embodiment, there is provided a method for protecting a subject from chemical warfare agents comprising (a) determining a chemical warfare threat; (b) providing to said subject an expression cassette comprising:

(i) a promoter active in host cells of said subject, and (ii) a gene encoding PON1 under the control of said promoter, in a form suitable for self administration; and

(c) providing to said subject information of said chemical warfare threat and instructions on the self administration of said expression cassette. The expression cassette may be in the form of a pharmaceutical preparation of a virus particle comprising said expression cassette, and the is PON1 type Q.

In still another embodiment, there is provided a method of protecting a subject from chemical warfare agents comprising administering to said subject an expression cassette comprising (a) a promoter active in cells of said subject; and (b) a gene encoding PON1 under the control of said promoter under conditions permitting expression of PON1. The expression cassette may be in the form of a pharmaceutical preparation of a infectious virus comprising said expression cassette. Again, the PON1 is PON1 type Q.

In still a further embodiment, there is provided a method of treating a subject to protect, correct or retard the progress of a neurodegenerative disease comprising administering to said subject an expression cassette comprising (a) a promoter active in cells of said subject; and (b) a gene encoding PON1 under the control of said promoter under conditions permitting expression of PON1. The neurodegenerative disease may be Parkinson's Disease or amyotropic lateral sclerosis.

In still yet another embodiment, there is provided a method of treating or protecting a subject from atherosclerosis comprising administering to said subject an expression cassette comprising (a) a promoter active in cells of said subject; and (b) a gene encoding PON1 under the control of said promoter under conditions permitting expression of PON1.

And in yet another embodiment, there is provided a method of treating or protecting a subject from Gulf War Syndrome comprising administering to said subject an expression cassette comprising(a) a promoter active in cells of said subject; and (b) a gene encoding PON1 under the control of said promoter under conditions permitting expression of PON1.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1.—Blood arylesterase activity increases after the administration of Ad.CMV-hPON1-LR or Ad.CMV-hPON1-LQ.

FIG. 2—Chlorpyrifos dose-response curve in mice.

FIG. 3A (top) & FIG. 3B (bottom)—Serum enzyme levels after administration of recombinant adenoviruses.

FIG. 4—Whole brain ACHE in mice after administration of recombinant adenoviruses and 30 mg/kg chlorpyrifos.

### DETAILED DESCRIPTION OF THE INVENTION

Five months after Iraq invaded Kuwait in 1990, the United States and its coalition forces launched Operation Desert Storm. It consisted of 5 weeks of bombing and 4 days of ground war. This war, though brief, exceeded the Vietnam War in the variety of potentially toxic environmental exposures. Large numbers of Gulf War veterans began to suffer from combinations of several symptoms. Studies have confirmed that the prevalence of symptoms is 2-3 times higher in Gulf War veterans than in military personnel not deployed to the war zone (Haley, 1999b).

Haley et al. (1999b) identified six unique symptom complexes representing neurologic syndromes or variants of neurologic injury sustained by military personnel in the Gulf War (Haley, 1999a). Ill veterans displayed results consistent with subcortical and brainstem dysfunction in neuropsychologic and audiovestibular tests. Three of the six complexes—impaired cognition, confusion-ataxia, athro-myoneuropathy—were strongly associated with risk factors for chemical exposures. The veterans in this study had self-reported exposures to combinations of OP pesticides, chemical nerve agents, high concentrations of DEET insect repellent, and symptoms of advanced acute toxicity after taking pyridostyigmine tablets (Haley, 1999a). Haley et al. (1999b) studied whether specific alleles or serum activity levels of PON1 may have put certain Gulf War veterans at higher risk for neurologic damage from environmental chemical exposure.

Blood was taken from ill and well subjects and was tested for PON1 genotype, phenotype and serum activity level. It was found that the veterans' health status was significantly associated with their PON1 polymorphism as well as their activity level. Ill veterans were more likely than well controls to have the R allele at position 192. They were also more likely to have lower arylesterase activity than well controls. The investigators plotted all subjects by their level of PON1 type Q activity and found a strong association between illness and having a low plasma level of type Q activity. Neither the type R level nor the polymorphism at position 55 was associated with the health status of the veterans (Haley, 1999a).

Many military personnel experienced similar environmental exposures and only some developed chronic illness. It is suggested that individuals with genetically determined low blood levels of type Q allozyme would allow more entry of the toxins to neural tissues and greater opportunity for neurotoxic damage. The results of this study further support the proposal that neurologic symptoms in some Gulf War veterans were caused by environmental chemical exposures and that genetic polymorphisms and serum levels of PON1 play an important role in senstivity to OP toxicity (Haley, 1999a).

As discussed above, serum activity levels and PON1 phenotype are associated with susceptibility to OP toxicity. Li et al. (1995) studied whether administering exogenous paraoxonase to mice would offer protection toward the acute toxicity of the OP chlorpyrifos. Paraoxonase was purified from rabbit serum and injected into mice. Chlorpyrifos was administered and inhibition of acetylcholinesterase (ACHE) in brain, diaphragm, plasma and red blood cells was measured to determine toxicity. It was found that rabbit paraoxonase protected against acetylcholinesterase inhibition and alleviated the signs of cholinergic intoxication (Li et al., 1995). This study employed purified rabbit paraoxonase, but no one has tested the ability of recombinant PON1 to provide protection.

This study is the first report of recombinant in vivo production of PON1 and its ability to protect animals from OP toxicity. Non-replicating adenoviruses which make recombinant PON1 (Q or R allozymes) in vivo were produced. The vectors were injected into mice. Sera from the mice were assayed for arylesterase and paraoxonase activities. The mice were challenged with chlorpyrifos and AchE levels in the brain were measured. The results indicated that a recombinant viral vector expressing paraoxonase can increase serum paraoxonase and arylesterase activity levels, and further, that this recombinant paraoxonase can protect mice from chlorpyrifos toxicity by preventing a dramatic decrease in whole brain acetylcholinesterase levels.

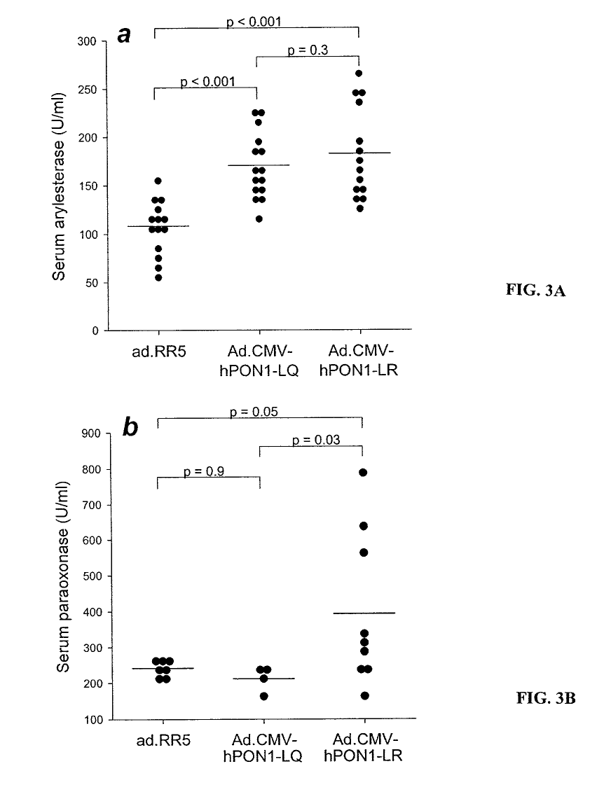

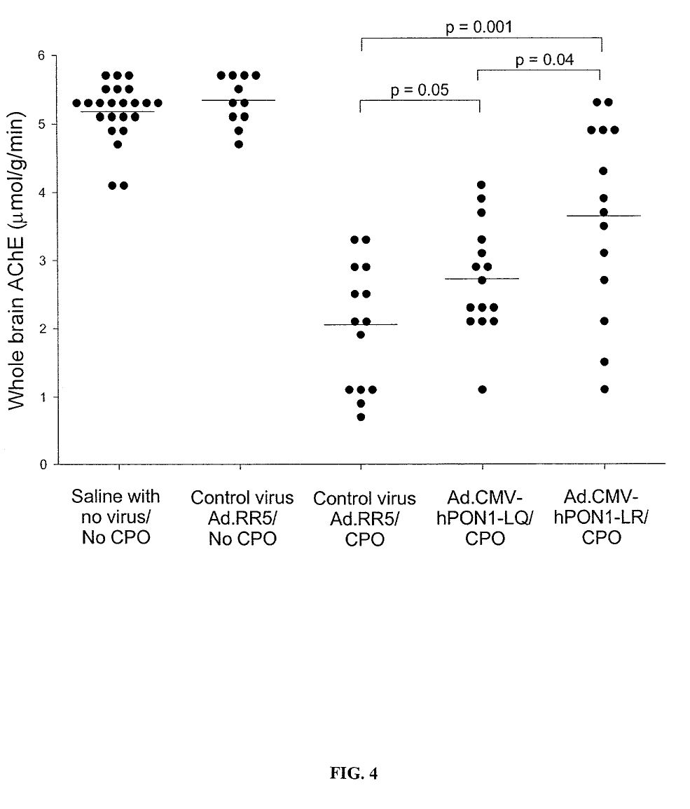

Both gene therapy vectors tested, Ad.CMV-hPON1-LR and Ad.CMV-hPON1-LQ, significantly increased serum arylesterase activity (FIG. 3A). However, only Ad.CMV-hPON1-LR increased serum paraoxonase activity (FIG. 3B). The LQ isoform has low activity against paraoxon, the substrate used to determine serum paraoxonase activity, and thus the inability of Ad.CMV-hPON1-LQ to increase serum paraoxonase levels was not surprising. Both PON-LR and PON-LQ provided significant protection against ACHE inhibition after chlorpyrifos challenge (FIG. 4). Serum arylesterase activity correlated with whole brain ACHE (FIG. 5).

The health of the animals is also noteworthy. Nine animals appeared sick after the 30 mg/kg chlorpyrifos challenge. Findings included mucus discharge from the eyes, lack of movement, and skin irritation. Sickness behavior was noted in five of the 15 (33%) mice that received Ad.RR5, one of the 15 (6%) of those that received Ad.CMV-hPON1-LR, one of the 15 (6%) that received Ad.CMV-hPON1-LQ, and one of the 5 (20%) that received saline. The average ACHE of the sick animals was 1.01±0.07 μmol/g/min. There appears to be a clinical correlation between the level of ACHE after chlorpyrifos challenge and the health status of the animal.

Thus, the results obtained here indicate that gene therapy can prevent organophosphate toxicity. Present treatment for organophosphate poisoning consists of post-exposure administration of a combination of drugs such as carbamates, antimuscarnics, reactivators and anticonvulsants (Gray, 1984). Doctor et al. (1991) have investigated using enzymes as pretreatment drugs to prevent OP toxicity and found that exogenously administered purified cholinesterases will act as scavengers that sequester OPs before they reach their physiological targets. However, gene therapy can provide a more complete response to OP toxin challenges.

I. PON1

The PON1 gene is located at q21-q22 on the long arm of chromosome 7 (Clendenning et al., 1996). The PON1 cDNA predicts a 355 amino acid protein. Human serum paraoxonase (PON1) has been purified to homogeneity (Gan et al., 1991). PON1 has a molecular mass of 43-45 kDa and contains up to three carbohydrate chains. The mature protein retains the hydrophobic signal sequence, with only the amino-terminal methionine residue being removed (Adkins et al., 1993; Hassett et al., 1991). The nucleotide and amino acid sequences are highly conserved (86% and 85%, respectively) in rabbit and human (Adkins et al., 1993). The human and mouse sequences of PON1 are also very similar (Sorenson et al., 1995a).

There are two structural isoforms of this enzyme. One isoform has all three cysteine residues free; the other has two cysteines engaged in a disulfide bond (Cys-41 and Cys-352), with the third being free (Cys-283). It was hypothesized that PON1 was a cysteine protease, but Sorenson et al. have shown that the free Cys-283 is not essential for activity (Sorenson et al., 1995b). The structure of the active site of PON1 has not been determined. It is known that Ca²⁺ is an essential cofactor for activity and stability of the enzyme. Ca²⁺ is important in maintaining the active site and protecting against enzyme inhibition by other metals (La Du, 1992).

PON1 is present in newborns and in premature infants. The level in infants is about half that of adults. Adult levels are reached one year after birth and usually remain unchanged (La Du, 1992). There have been no differences detected in PON1 activity levels between sexes (Playfer et al., 1976).

It is thought that PON1 is synthesized and secreted by the liver. Northern blot analyses of various tissues detected PON1 mRNA only in liver (Hassett et al., 1991). Reverse transcriptase polymerase chain reaction (RT-PCR), a more sensitive technique, has shown this mRNA in mouse liver, lung, heart, brain, small intestine, and kidney (Primo-Parmo et al., 1996). It is not certain which, or how many, tissues contribute to the PON1 activity found in serum.

The natural substrate and physiological role of PON1 are not known at this time. It is true that PON1 is important in metabolizing OPs, but most of these compounds are not found in nature. It is likely that they are hydrolyzed by PON1 simply due to their structure (Mackness et al., 1998). Experiments continue to be performed to determine this enzyme's natural substrate.

II. PON1 Polymorphisms

There are two known polymorphisms in the PON1 gene. The polymorphism at amino acid residue 192 affects the activity of the enzyme toward certain substrates. The Q (formerly A) isoenzyme has glutamine at position 192 and has low activity toward paraoxon. The R (formerly B) isoenzyme has arginine at this position and has high activity toward paraoxon (Adkins et al., 1993). Both isoforms hydrolyze phenylacetate at approximately the same rate. It has recently been found that other substrates such as diazinon, sarin, and soman are discriminatory in a reverse manner compared to paraoxon. The Q alloenzyme hydrolyzes these rapidly and the R alloenzyme does so slowly (Davies et al., 1996). The Q alloenzyme hydrolyzes chlorpyrifos at approximately 75% of the rate of the R alloenzyme (Davies et al., 1996).

The other polymorphism is at amino acid residue 55 and is a leucine-to-methionine (L or M) substitution. The importance of this polymorphism is less clear, yet it has been suggested that it also affects activity. The M allele seems to be associated with less PON1 activity than the L allele (Mackness et al., 1997).

Genetic studies of these polymorphisms have shown that PON1 activity in Europeans is inherited as a simple Mendelian trait. It is determined by two alleles operating at a single autosomal locus following Hardy-Weinberg principles (Playfer et al., 1976). Populations in Africa and the Orient and the Canadian Inuit show a loss of the low activity phenotypes (types Q and A) and a unimodal distribution (La Du, 1992).

The genetic polymorphisms of the PON1 gene affect activity toward some substrates. It has been proposed that they affect concentration as well. Serum activity in healthy patients showed a direct correlation to protein concentration (Mackness et al., 1998). It has been suggested that genotyping for these polymorphisms may provide a basis for determining a person's susceptibility to OP poisoning (Costa L G & Manzo, 1995). A current view is that the serum concentration of the R and Q isoenzymes will determine susceptibility to different OPs. High concentrations of Q will protect against sarin, soman, and diazinon; whereas, high concentrations of R will protect against parathion, and other pesticides (Haley et al., 1999a).

III. Vectors for Delivery of PON1

Within certain embodiments expression vectors are employed to express a PON1 polypeptide product. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” is meant to include any type of genetic construct containing a nucleic acid coding for a gene product in which part or all of the nucleic acid encoding sequence is capable of being transcribed and translated into a polypeptide product. An “expression cassette” is defined as a nucleic acid encoding a gene product under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy | Banerji et al., 1983; Gilles et al., 1983; Gros- |
| Chain | schedl et al., 1985; Atchinson et al., 1986, |
| | 1987; Imler et al., 1987; Weinberger et al., |
| | 1984; Kiledjian et al., 1988; Porton et al.; 1990 |
| Immunoglobulin Light | Queen et al., 1983; Picard et al., 1984 |
| Chain | |
| T-Cell Receptor | Luria et al., 1987; Winoto et al., 1989; |
| | Redondo et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins (GRP94 and | |
| GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., 1981; |
| | Sleigh et al., 1985; Firak et al., 1986; Herr et |
| | al., 1986; Imbra et al., 1986; Kadesch et al., |
| | 1986; Wang et al., 1986; Ondek et al., 1987; |
| | Kuhl et al., 1987; Schaffner et al., 1988 |
| Polyoma | Swartzendruber et al., 1975; Vasseur et al., |
| | 1980; Katinka et al., 1980, 1981; Tyndell et al., |
| | 1981; Dandolo et al., 1983; de Villiers et al., |
| | 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Choi et al., 1988; Reis- |
| | man et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987; Glue et al., 1988 |
| Hepatitis B Virus | Bulla et al., 1986; Jameel et al., 1986; Shaul et |
| | al., 1987; Spandau et al., 1988; Vannice et al., |
| | 1988 |
| Human | Muesing et al., 1987; Hauber et al., 1988; Jak- |
| Immunodeficiency Virus | obovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foeck- |
| | ing et al., 1986 |
| Gibbon Ape Leukemia | Holbrook et al., 1987; Quinn et al., 1989 |
| Virus | |

TABLE 2
Inducible Elements

| Element | Inducer | References |
|---|---|---|
| MT II | Phorbol Ester | Palmiter et al., 1982; Haslinger |
| | (TFA) | et al., 1985; Searle et al., 1985; |
| | Heavy metals | Stuart et al., 1985; Imagawa |
| | | et al., 1987, Karin et al., 1987; |
| | | Angel et al., 1987b; McNeall |
| | | et al., 1989 |
| MMTV | Glucocorticoids | Huang et al., 1981; Lee et al., |
| (mouse mammary | | 1981; Majors et al., 1983; |
| tumor virus) | | Chandler et al., 1983; Lee et al., |
| | | 1984; Ponta et al., 1985; Sakai |
| | | et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| CRP | IL-6, IL-1 | Ku & Mortensen, 1993 |
| SAA | IL-6, IL-1 | Jiang et al., 1995 |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large | Taylor et al., 1989, 1990a, |
| | T Antigen | 1990b |
| Proliferin | Phorbol Ester- | Mordacq et al., 1989 |
| | TPA | |
| Tumor Necrosis | TPA | Hensel et al., 1989 |
| Factor | | |
| Thyroid Stimulating | Thyroid Hormone | Chatterjee et al., 1989 |
| Hormone α Gene | | |

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

(iv) Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

(v) Delivery of Expression Vectors

Viruses:

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Adenovirus:

One of the methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Retrovirus:

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Adeno-Associated Viruses:

Adeno-associated virus (AAV) is an attractive virus for delivering foreign genes to mammalian subjects (Ridgeway, 1988; Baichwal and Sugden, 1986; Hermonat and Muzycska, 1984). AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The sequence of AAV is provided by U.S. Pat. No. 5,252,479 (entire text of which is specifically incorporated herein by reference).

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector of the present invention can be obtained by restriction endonuclease digestion of AAV or a plasmid such as p201, which contains a modified AAV genome (Samulski et al., 1987). Alternatively, the terminal repeats may be obtained by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

Other Viruses:

Other viral vectors may be employed as expression constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Non-Viral Methods:

Several non-viral methods for the transfer of expression constructs into mammalian cells also are contemplated by the present invention. These include DEAE-dextran (Gopal, 1985), electroporation (Tur-Kaspa et al., 1986; Potter et al., 1984), direct microinjection (Harland and Weintraub, 1985), lipofectamine-DNA complexes, cell sonication (Fechheimer et al., 1987), and receptor-mediated transfection (Wu and Wu, 1987; Wu and Wu, 1988).

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Liposomes:

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are Lipofectamine®-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells.

IV. Methods for Diagnosing, Preventing and Treating Organophosphate Toxicity

(i) Diagnosing PON1 Status

The plasma concentrations of the Q and R allozymes of the PON1 enzyme, as well as the plasma concentration of butyrylcholinesterase, are the body's main intrinsic determinants of susceptibility to organophosphate (OP) toxicity (Broomfield et al., 1991; Doctor et al., 1993; Loewenstein-Lichtenstein et al., 1995; Li et al., 1993; Shih et al., 1998; Costa et al., 1999; Haley et al., 1999). They also play a potentially important role in preventing accelerated atherosclerosis, retinopathy and neuropathy, particularly in non-insulin-dependent diabetes mellitus, by preventing peroxidation of low-density lipoprotein (LDL) (Mackness et al., 1998; Pfohl et al., 1999; Sakai et al., 1998; Odawara et al., 1997; Kao et al., 1998; Cao et al., 1999; Ikeda et al., 1998). Although the presence of the Q and R allozymes is determined by one's PON1 genotype, an as yet unknown promoter-enhancer gene(s) plays a role in determining the plasma concentrations of the allozymes. Consequently, measurement of plasma allozyme concentrations is important to identify PON1-deficient individuals; whereas, genetic analysis is not presently as important (Haley et al., 1999).

Although the analytical methods for measuring the plasma concentrations total paraoxonase activity (Eckerson et al., 1983), total arylesterase activity (Lorentz et al., 1979) and related enzymes were described long ago, no method for measuring the concentrations of the Q and R allozymes were known until recently. From the measured values of total paraoxonase activity and total arylesterase activity, one can deduce the Q/R phenotype and calculate the proportion (and concentrations) of total arylesterase activity due to the Q and R allozymes (Haley et al., 1999; Eckerson et al., 1983).

Basically, the Q/R phenotype is inferred from the ratio of total paraoxonase activity to total arylesterase activity (the P/A ratio), which typically shows three clusters of test subjects in any human population (Haley et al., 1999). Values of the P/A ratio around 1.2 indicate Q homozygotes, values around 9.0 indicate R homozygotes, and values around 3.9 indicate QR heterozygotes (Haley et al., 1999). For the homozygous Q individuals, the Q allozyme concentration equals the total arylesterase concentration, and their R allozyme level is zero. For the homozygous R individuals, the R allozyme concentration equals the total arylesterase concentration, and their Q allozyme level is zero. For the heterozygous individuals, the total arylesterase concentration is apportioned into Q and R components by interpolation from the P/A ratio as follows:

$$Proportion Type R = \frac{9.00 - P / A ratio}{7.8}$$

$$Proportion Type Q = 1.00 - \frac{9.00 - P / A ratio}{7.8}$$

Each of these proportions is multiplied by the total arylesterase concentration to obtain the Q and R allozyme concentrations.

A problem with the above enzymatic assays is that they involve antiquated test-tube enzymatic techniques, which use toxic reagents and require highly experienced research technicians to perform. This makes the test time consuming and expensive. Generally, only a few tests can be run at a time, and quality control is a problem. Recently, the inventors' laboratory at the University of Texas Southwestern Medical Center developed a rapid, fully automated assay using a Chem Well™ Analyzer. The protocol can be adapted to Olympus or other major autoanalyzers including the new IGEN rufenium chemi-luminescence technology. Any of these can be used for cost-effective mass testing of PON1 Q and R allozyme concentrations. The inventors have developed similar rapid automated assays for plasma cholinesterase, including dibucaine and fluoride inhibition numbers. Consequently, rapid, large scale testing for PON1 Q and R allozyme levels is now for the first time practical.

(ii) Preventing Organophosphate Toxicity

Current standard approaches for preventing organophosphate (OP) toxicity in warfare situations involve a combination of chemical weapon (CW) detection and avoidance (U.S Senate Committee Report, 1994). Military units anticipating CW nerve agent exposure carry OP detection devices that sound an alarm when traces of OP agents are detected in ambient air. When alerted by an OP alarm, soldiers don MOPP (mission oriented protective posture) protective clothing, which includes a charcoal-lined impervious rubber body suit, gloves, boots, and gas mask. MOPP gear has a finite protective life, becoming ineffective after hours of CW exposure, requiring the gear to be replaced for repeated exposures. This system is very effective in protecting from a one-time CW exposure but, as shown in the Gulf War, has limitations for repeated exposure or low-level exposure situations. The need to replace MOPP gear in repeated-exposure settings produces problems in resupply and in the risk of exposure in handling contaminated MOPP gear.

The biggest deficiency is in protection from low-level CW exposure where either ambient OP concentrations are below the sensitivity thresholds of OP detection equipment or where repeated low-level exposures without acute casualties are disregarded by military commanders as “false alarms.” Both of these situations occurred repeatedly during the air war, ground war and cleanup phases of the Gulf War (U.S. Senate Committee Report, 1994; Tucker, 1997). Evidence from two epidemiologic studies has implicated low-level CW exposure as a potential cause of chronic brain damage in Gulf War veterans (“Gulf War syndrome”). Haley et al., studying a Naval Reserve battalion, found that veterans with Gulf War syndrome were 7.8 times more likely to report indicators of low-level CW exposure than well veterans (p =0.001) (Haley R W & Kurt T L, 1997). Kang et al., studying a random sample of approximately 20,000 Gulf War veterans, reported the same finding with a similar relative risk, 7.1 times (Kang et al., 1999). Similarly, Japanese physicians have reported chronic brain damage in survivors of the terrorist attack with sarin nerve agent in the Tokyo and Matsumoto subways (Murata et al., 1997; Yokoyama et al., 1998; Himuro et al., 1998). Animal studies have shown that repeated, low-level sarin exposure produces chronic neurologic damage (Husain et al., 1995; Husain et al., 1993). With this information becoming increasingly known, it seems likely that future military adversaries and domestic terrorists will use low-level CW, or the threat of it, in military or terrorist situations.

A ready solution to all of these threats is to boost the blood level of PON1 enzymes, particularly the Q allozyme, in military personnel, police, anti-terrorist units and other high risk groups to boost this natural barrier against low-level CW agents. In the Gulf War, despite the fact that large numbers of military personnel were exposed to low-level CW agents, relatively few (approximately 10% to 15%) became ill, and initial evidence indicates that a genetically determined low blood level of the Q allozyme was the reason (Haley et al., 1999). This finding has recently been confirmed and applied more specifically to CW agents by Broomfield at the Biochemical Pharmacology Branch, U.S. Army Medical Research Institute of Chemical Defense, who tested the same blood specimens against sarin and soman as substrates for the enzyme assay.

The PON1 Q allozyme hydrolyzes sarin and soman in blood before it can reach brain or fat tissue. The fact that the dose-response curves for OP toxicity are steep (Coata et al, 1990) predicts that small differences in hydrolytic rates (enzyme concentrations) below a critical threshold should account for large differences in toxicity (Davies et al., 1996). This means that boosting PON1 allozyme blood levels even small amounts should protect from low concentrations of CW agents, and boosting it by larger amounts should protect against even higher CW concentrations. This phenomenon has been demonstrated in rodents (Li et al., 1995).

Therefore, prophylactic boosting of PON1 allozyme concentrations with gene therapy to different levels will depend on intrinsic PON1 enzyme levels and CW threat levels. First, military troops will be tested for intrinsic PON1 enzyme levels, and those with low blood levels of the Q allozyme (below 80 units/L) (Haley et al., 1999) will receive gene therapy to boost their blood levels above that critical threshold to protect against low-level CW threat. Second, when facing credible threats of high level CW attacks, all troops could receive gene therapy to boost their blood levels of the Q allozyme to levels far above the usual normal range, thereby conferring extraordinary protection from much higher CW concentrations. It is conceivable that high enough blood levels could be achieved to render subjects safe from massive CW attacks.

A refinement that the inventors have planned is to include a regulatory gene along with the PON1 gene in the gene therapy that would allow on-off regulation of PON1 enzyme production. This gene combination would be introduced on a vector that would produce long-lasting or permanent persistence of the new genetic material in the host. The regulatory gene would be turned on by some benign medication (the “elicitor” compound) to which personnel are unlikely to be exposed otherwise (e.g., a unique tetracycline derivative). Thus, the PON1 gene inserted in the persistent vector would remain perpetually nonfunctional; however, when faced with a situation where CW exposure was likely, personnel would begin taking the elicitor compound, which would turn on PON1 Q (or R) allozyme production as long as the elicitor was being taken. In this way personnel would be protected by increased PON1 Q allozyme production only during brief periods when it was needed. The inventors also envision the model being developed so that the dose of the elicitor compound would determine the level of PON1 Q allozyme production, or different elicitor compounds would turn on the PON1 gene(s) to produce low or high PON1 Q allozyme expression. In this way, personnel could be protected from low or high-level CW exposures by boosting the body's own natural protective mechanism to the level required by the level of the CW threat.

The same technology will also be broadly applicable to protecting agricultural or industrial workers from occupational exposures to OP pesticides, lubricants, fumes, etc.

(iii) Treating Organophosphate Toxicity

Current state-of-the-art technology for treating OP exposures is to inject or infuse atropine to counter the cholinergic symptoms (particularly the life-threatening pulmonary secretions) and pralidoxime (2-PAM) to attempt to split the CW molecules off the acetylcholinesterase (ACHE) enzyme molecules to reconstitute their activity (Caroscio et al., 1987). The latter is only effective until the CW agent has undergone “aging,” a process that makes the CW-ACHE bond and the inactivation of AChE permanent. This interval varies by CW agent, for example, five hours for sarin (GB) but only two minutes for soman (GD) (Dunn Sidell, 1989). Moreover, the atropine-pralidoxime infusion treatment may have to be continued in an intensive care unit (ICU) setting for many days in cases where CW exposure was heavy (Sidell, 1974). During this treatment phase, CW molecules initially absorbed into the body's fat stores continue to be released back into the blood where they can circulate to lung and nervous system tissue and threaten life. Infusion treatment must be continued until CW stores are eliminated.

In the Gulf War, the carbamate medication pyridostigmine bromide (PB) was used as a pre-exposure antidote to increase the efficacy of the post-exposure atropine-pralidoxime treatment (Keeler et al., 1991). Administration of a 30 mg PB tablet three times a day had been shown in animal experiments to increase the efficacy of the post-exposure treatment of a soman (GD) exposure by 50%. Increasing evidence since the Gulf War, however, has implicated PB as a contributor to the brain damage underlying Gulf War syndrome (Haley & Kurt, 1997; Abou-Donia et al., 1996; Li et al., 2000). Despite continuing research, it remains to be seen whether pre-exposure chemo-prophylaxis will prove safe and effective.

Consequently, there is a void in technology for treating casualties of CW attacks, particularly when the rapidly aging agent soman (GD) is involved. Boosting of the PON1 Q allozyme will provide a valuable new adjunct to treatment. Boosting PON1 Q allozyme blood levels in the period between CW exposure and the completion of aging will contribute to hydrolyzing and eliminating much of the CW agent as it circulates in plasma and reaches a steady state of dissociation between plasma, butyrylcholinesterase, and ACHE receptors. This could be accomplished by administering the elicitor compound in CW-exposed personnel who were previously treated with the PON1 gene therapy. In the stage of subacute ICU treatment, high PON1 Q allozyme levels may have an important role in reducing the duration of treatment by inactivating CW molecules as they come out of fat stores into blood before they enter the nervous system and neuromuscular junction. In this stage, over and above administration of the elicitor compound to previously gene-therapy-treated personnel, there might be sufficient time for de novo gene therapy to contribute in personnel not previously treated with gene therapy.

These techniques could be used as well for treatment of civilians exposed to high levels of OPs in occupational settings.

(iv) Preventing and Treating Atherosclerosis

The causes of many neurodegenerative diseases, such as Parkinson's disease (PD) and amyotrophic lateral sclerosis (ALS or Lou Gehrig's disease), remain largely unknown. Emerging evidence, however, is increasingly pointing toward progressive brain cell damage from environmental toxins interacting with genetic susceptibilities in rare individuals (Poirier et al., 1991; Checkoway et al., 1998; Langston, 1998).

For example, PD and ALS have been occurring in epidemic form on certain Pacific islands since the 1950s, suggesting an environmental toxin in genetically inbred populations (Oyanagi & Wade, 1999; McGeer et al., 1997). Epidemiologic studies have shown an association between PD and lifetime pesticide exposure (Le Couteur et al., 1999), PD has been found to occur more commonly in people with a certain genotype of the paraoxonase PON1 enzyme which protects against pesticides and other organophosphate toxins (Konda & Yamamoto, 1998), and PD has recently been produced in rats by chronic treatment with a pesticide (Betarbet et al., 2000).

The only risk factor that is strongly associated with ALS in a dose-response manner is a history of working as a farmer or other occupational exposure to pesticides (Bharucha et al., 1983; McGuire et al., 1997). The present inventors have described an epidemic of ALS in young Gulf War veterans, most of whom had Gulf War syndrome shortly after the war and before developing ALS. In addition, it has been found that that the Gulf War veterans with ALS have significantly lower levels of the plasma butyrylcholinesterase and/or the PON1 paraoxonase Q allozyme, the genetically controlled blood enzymes that protect against organophosphate toxicity. Although not yet constituting conclusive evidence of a causal connection, chronic neurotoxicity from organophosphate exposure is one of the most strongly supported theories of the cause of these neurodegenerative diseases.

Given the demonstrated role of paraoxonase and its allozymes as a risk factor in accelerated atherosclerosis and neurologic complications of non-insulin-dependent diabetes mellitus (Mackness et al., 1998; Pfohl et al., 1999; Sakai et al., 1998; Odawara et al., 1997; Kao et al., 1998; Cao et al., 1999; Ikeda et al., 1998), it is possible that screening for low PON blood levels in diabetics, and others at risk for accelerated atherosclerosis, retinopathy or neuropathy or in those with premature complications of these processes, and boosting their blood levels with PON1 gene therapy might prevent further progression and complications. This is becoming an increasingly important issue because of the current epidemic of type II, non-insulin-dependent diabetes mellitus that is sweeping the U.S. in the 1990s and early 2000s. Thus, PON enzyme screening and gene therapy are useful in preventing atherosclerosis, retinopathy and neuropathy in diabetics as well in patients with other conditions with accelerated atherosclerosis, retinopathy and neuropathy. The PON screening and gene therapy issues would be managed in these additional groups similarly to the measures described above for groups at risk of exposure to and injury from organophosphates.

(v) Kits for Administering PON1 Vectors

The present invention also provides therapeutic kits. Such kits will generally contain, in suitable container means, a pharmaceutically acceptable formulation of vector encoding PON1 in a form suitable for administration to a subject. The kits may also contain other pharmaceutically acceptable formulations, such as buffers or agents that increase gene uptake or expression.

The kits may have a single container means that contains the expression construct in a form suitable for administration. Other kits of the present invention include the expression construct in a storage stable form, along with buffers or diluents in separate and distinct containers. For example, when the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container means.

The container means of the kit may also include at least device for administration of the expression construct. For example, a syringe or inhaler may be included. In some embodiments, the expression construct may be pre-mixed and aliquoted into a unit dosage form and loaded into such a device. The kits may contain multiple devices for repeat administration or administration to more than one subject.

The kits of the present invention will also typically include a means for containing the vials, devices or such in close confinement for shipment, storage or commercial sale, such as, e.g., injection or blow-molded plastic containers into which the desired vials and other apparatus are placed and retained. The kits also may contain instructions for administration, including self-administration.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Culture:

Human liver HepG2 cells and human embryonic kidney cells were obtained from ATCC. The cells were grown in DMEM (GIBCO-BRL, Grand Island, N.Y.) supplemented with 10% heat-inactivated fetal calf serum (Summitt Biotechnology, Fort Collins, Colo. or Atlanta Biologicals, Norcross, Ga.), 50 U/ml penicillin G, and 50 μg/ml streptomycin in a 5% CO₂ atmosphere at 37° C.

Animals:

Eight-week-old male ICR and BALB/c mice were obtained from Harlan Farms (Indianapolis, Ind.). They were housed at 20° C. and were given food and water ad libitum. They weighed between 19-28 grams at the time of procedures.

Isolation of PON1 RNA, Production of cDNA, PCR of Gene:

Total RNA was isolated from HepG2 cells using the Ultraspec RNA isolation system (Biotex Labs, Houston, Tex.). cDNA was made from total RNA using random hexamers or oligoDT and Moloney Murine Leukemia Virus Reverse Transcritpase (M-MLV RT). The PON1 gene was amplified from the cDNA using Taq polymerase, primer III.66 (5′-GCG GCC GCA TGG CGA AGC TGA TTG CGC TCA CCC TCT) and primer III.80 (3′-TCT AGA TTA GAG CTC ACA GTA AAG AGC TTT GTG AAA). This generated a 1068 base pair fragment (the PON1 cDNA) with NotI (5′) and XbaI (3′) ends. PCR and restriction analysis or automated sequencing can be performed to determine the polymorphisms at amino acid positions 55 and 192 (Humbert et al., 1993). PCR and restriction analysis were used to determine if residue 192 was an arginine or a glutamine. A 100-base pair fragment spanning residue 192 was generated using primers III.60 (5′-TAT TGT TGC TGT GGG ACC TGA G) and III.61 (3′-CAC GCT AAA CCC AAA TAC ATC TC). This fragment was digested with AlwI. AlwI cleaves the fragment if residue 192 is an arginine (CGA), but not if it is a glutamine (CAA). Automated sequencing was performed to confirm this and to determine if amino acid residue 55 was a leucine (TTG) or a methionine (ATG). The PON1 gene isolated from HepG2 cells and amplified was Leu55 and Arg192 (PON1-LR).

Cloning and Mutagenesis of the PON1 Gene:

pZero2.1 (Invitrogen, Carlsbad, Calif.) was digested with Ecl1361II and ligated to the PON1-LR PCR product to produce clone pZero-hPON1-PCR. This plasmid and pGem11Zf (Promega, Madison, Wis.) were digested with EcoRI and HindIII. The PON1-LR gene was isolated from pZero-hPON1-PCR and cloned into pGem11Zf producing plasmid pGem11-hPON1-LR. pGem11Zf is the vector used in the GeneEditor in vitro Site-Directed Mutagenesis System (Promega). Site-directed mutagenesis was performed to mutate Arg192 to Gln192 using pGem11-hPON1-LR as the template, oligo 111.73 (ACC CCT ACT TAC AAT CCT GGG AG), and T4 DNA polymerase. The mutated region of several clones was sequenced to verify the mutation. One LQ clone was then completely sequenced ensure that additional mutations had not been introduced. The resulting clone, pGem11-hPON11-LQ, was used in all subsequent manipulations.

PON1-LR and PON1-LQ inserts were removed by digestion with Asp718 and XbaI and moved into pAC.CMVpLpA (gift of Robert Gerard) digested with the same enzymes. The resulting plasmids, pAC.CMV.hPON1-LR and pAC.CMV-hPON1-LQ, were used to produce recombinant adenoviruses.

Production of Recombinant Adenovirus:

Ad.CMV-hPON1-LR and Ad.CMV-hPON1-LQ were produced by homologous recombination in 293 cells by the following method. A freshly confluent plate of 293 cells was split 1:4 and allowed to grow until 80 to 90% confluent. They were then cotransfected with 15 μg of pAC.CMV.hPON1-LR or pAC.CMV.hPON1-LQ and 1 μg of XbaI-digested viral genomic DNA from Ad. gal (gift of Dr. Robert Gerard) using Superfect transfection reagent (Boehringer Mannheim, Indianapolis, Ind.). After plaques had appeared, the plates were overlayed with semisolid agarose (0.5% agarose in 1× PBS) containing Xgal (1 mg/ml). White plaques were cored, transferred to 150 μl of infection medium (DMEM supplemented with 2% heat inactivated FCS, penicillin and streptomycin) and soaked overnight. Fifty μl of the resulting lysates were then used to infect freshly confluent cultures of 293 cells in 48-well plates. After four to five days, the supernatants were harvested and tested for PON activity by the phenylacetate assay (described below). Two positive clones of each virus were then subjected to three cycles of plaque isolation and screening to ensure purity. They were grown to high titer in 293 cells, harvested, purified over a discontinuous CsCl gradient, and desalted on a Sepharose CL-4B (Pharmacia, Piscataway, N.J.) column as described (Gerard & Meidell, 1995). After the addition of low-endotoxin bovine serum albumin (10 μg/ml), aliquots were quick frozen and stored at −70° C. until used. The virus titer was then determined by plaque assay on 293 cell monolayers (Green, 1979).

Ad.RR5 (gift of Dr. Robert Gerard) and Ad.CMV-luc were used as negative controls.

Phenylacetate Assay:

Determination of serum arylesterase was performed according to the method of Lorentz et al. (Lorentz et al., 1979). Briefly, 5 μl of serum was added to 200 μl of activator solution (20 mM CaCl₂ and 155 mM NaCl). 2.2 μl of the diluted sample was combined with 220 μl of reagent 1 (54.4 mM Tris acetate, pH 7.5, 0.56 mM 4-aminoantipyrine, and 4.8 mM phenylacetate) and incubated for 5 minutes. The differential absorbance was then measured at 545 nm in a ChemWell Spectrophotometer (Awareness Technologies, Palm City, Fla.) at 37° C. 17 μl of 85.2 mM potassium ferricyanide was added and incubated for 5 more minutes. Then, a final absorbance was taken and the results were calculated yielding U/mL. One unit of arylesterase activity is defined as 1 μmole phenylacetate hydrolyzed per minute.

Administration of Virus and Drug to Animals:

Each animal was injected via the tail vein with a single adenovirus construct or saline. Ad.RR5, Ad.CMV-hPON1-LR, and Ad.CMV-hPON1-LQ were diluted in sterile 0.9% saline to 2×10⁹ pfu/200 μl. Each animal was injected with 200 μl virus/saline or saline alone. Chlorpyrifos (Chem Service, West Chester, Pa.) was prepared in DMSO. Different doses (0, 1, 2, 5, 10, 20, 30, 40, 80, 160 mg/kg) were injected s.c. into the mice.

AChE Determination:

18-24 hours after drug administration, mice were anesthetized with halothane (Sigma Chemical, St. Louis, Mo.), transcardially perfused with sterile 0.9% saline, decapitated, and whole brains removed. Any remaining brain stem was removed and the rest of the brain was homogenized. Determination of whole brain AChE was performed according to the method of Ellman et al. (1961). Briefly, 150 mg of brain homogenate was added to 1.5 ml of Tris buffer, 0.05 M, pH 8.0, maintained at 25° C., and rehomogenized. Fifty μl of the resulting homogenate was then combined with 3.2 ml of Tris buffer, 0.05 M, pH 7.4, with 0.25 μM DTNB (5,5′-dithio-bis[2-nitrobenzoic acid]), also maintained at 25° C. in a cuvette and stirred. A double beam spectrophotometer (Lambda 12; Perkin-Elmer, Norwalk, Conn.) was used to derive the absorbance slope, and hence enzyme activity, of the sample. One hundred μl of the substrate, 0.156 M acetlythiocholine iodide freshly prepared in distilled water, was added to the cuvette and absorbance at 405 nm was determined 1 and 3 minutes later. All samples were determined in duplicate and the average was taken as enzyme activity. ACHE is measured as micromoles of thiocholine produced per gram of homogenized tissue per minute (μmol/g/min).

Paraoxonase Assay:

Serum paraoxonase activity was determined as described by Eckerson et al (1983). 300 μl of buffer/substrate reagent (50 mM glycine buffer with 1M NaCl, pH 10.5, 1 mM CaCl₂, and 0.25 mM paraoxon in 20 mM NaCl) was placed in a cuvette and the absorbance taken in a ChemWell Spectrophotometer (Awareness Technologies) at 405 nm. 3 μl of the serum sample was then added to the cuvette and incubated for 10 minutes at 37° C. A final absorbance reading was then taken, and results determined yielding U/mL. One unit of paraoxonase activity is defined as 1 nmole 4-nitrophenol formed per minute.

Statistical Methods:

The main hypothesis of protection from inactivation of ACHE by chlorpyrifos was tested globally by a nonparametric k-sample test of independence of the whole brain ACHE activity and the three experimental groups using the Krustal-Wallis test. After rejecting the global null hypothesis, individual differences among the groups were tested with the Mann-Whitney U two-sample test using a one-tailed test under the assumption that increased serum arylesterase could only increase or fail or affect the whole brain AChE activity. Group differences in serum arylesterase and paraoxonase activity were tested with a similar strategy.

Simple linear regression was used to test the association of the final serum arylesterase activity and whole brain AChE activity in all groups combined. All deviations are expressed as ±SEM.

### Example 2

### Results

Testing Recombinant Adenoviruses in vitro:

The inventors used 293 cells to test recombinant adenoviruses in vitro. 2.5×10⁶ cells were infected with Ad.CMV-luc (negative control), Ad.CMV-hPON1-LR, or Ad.CMV-hPON1-LQ at a multiplicity of infection of 5 (or 1.25×10⁷ pfu) in duplicate. Cells and the overlying media were harvested at 48 hours post infection. The cells were lysed in 500 μl of detergent-based buffer and assayed for arylesterase activity along with the media. The results are shown in Table 3.

TABLE 3
Testing Ad.CMV-hPON1-LR and Ad.CMV-hPON1-LQ in vitro

Arylesterase U/ml

| Virus | Media | Cell Lysates |
|---|---|---|
| Ad.CMV-luc | −0.1 | −0.1 |
| | −0.2 | −0.1 |
| Ad.CMV-hPON1-LR | 7.5 | 0.9 |
| | 6.6 | 0.8 |
| Ad.CMV-hPON1-LQ | 8.1 | 7.6 |
| | 8.1 | 7.8 |

Both recombinant adenoviruses produced a protein which had arylesterase activity. Most of the cells that were infected with Ad.CMV-hPON1-LR had lifted off the dish and lysed, not leaving many cells to prepare cell lysates. This could explain why little activity was present in the Ad.CMV-hPON1-LR cell lysates.

Preliminary Testing of Recombinant Adenoviruses in vivo:

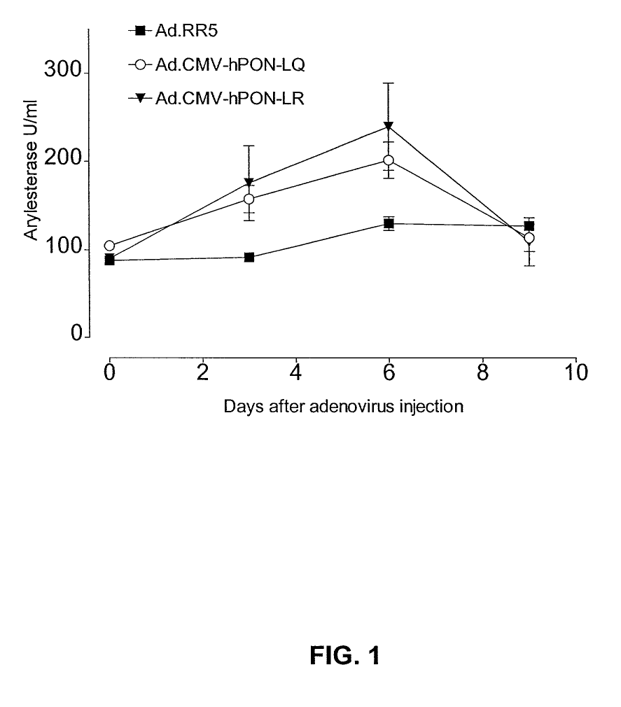

The inventors wanted to test whether Ad.CMV-hPON1-LR and Ad.CMV-hPON1-LQ would increase the serum level of arylesterase in mice. Blood was taken from three ICR mice before treatment with viruses to determine the baseline range of serum arylesterase. On Day 0, mice were injected i.v. with 2×10⁹ pfu of Ad.RR5 (negative control), Ad.CMV-hPON1-LR, or Ad.CMV-hPON1-LQ. On Day 3, Day 6, and Day 9, blood was collected from the mice and sera were assayed for arylesterase. The results are shown in Table 4 and FIG. 1.

TABLE 4
Testing Ad.CMV-hPON1-LR and Ad.CMV-hPON1-LQ in vivo

| | Arylesterase U/ml | | | |
|---|---|---|---|---|
| Virus | Day 0 | Day 3 | Day 6 | Day 9 |
| Ad.RR5 | 87.7 | 90.0 | 128.1 | 123.3 |
| | | 83.1 | 143.1 | 134.3 |
| | | 93.2 | 139.3 | — |
| | | 82.5 | — | — |
| | | 108.4 | 108.0 | 122.8 |
| mean ± SEM | | 91.4 ± 4.7 | 129.6 ± 7.9 | 126.8 ± 3.8 |
| Ad.CMV- | 90.1 | 275.8 | 339.1 | 96.1 |
| hPON1-LR | | 97.7 | 150.2 | 76.6 |
| | | 77.3 | 99.4 | 90.5 |
| | | 153.5 | 264.7 | 65.3 |
| | | 272.5 | 343.1 | 215.8 |
| mean ± SEM | | 157.3 ± 15.4 | 201.5 ± 20.6 | 113.4 ± 15.5 |
| Ad.CMV- | 104.6 | 149.1 | 167.6 | 158.0 |
| hPON1-LQ | | 101.8 | 142.2 | 136.8 |
| | | 190.0 | 265.0 | 71.3 |
| | | 206.2 | 260.4 | 108.9 |
| | | 160.8 | 195.7 | — |
| | | 135.8 | 178.0 | 92.2 |
| mean ± SEM | | 175.4 ± 42.2 | 239.3 ± 49.4 | 108.9 ± 27.3 |
| Baseline | 94.1 ± 5.28 | | | |
| mean ± SEM | | | | |

Administration of 2×10⁹ pfu of Ad.CMV-hPON1-LR or Ad.CMV-hPON1-LQ into the tail veins of mice increased their levels of serum arylesterase over that of the controls. The protein was present by the third day after virus administration, was still being produced at Day 6, and was no longer produced on Day 9. This time course of recombinant protein expression was similar to that found previously for other proteins made from this adenoviral vector (Coulthard et al., 196). Based on this result, a 3-day trial was chosen after viral injection to measure enzyme activity and day 4-5 for drug challenge.

Chlorpyrifos Dose-Response Curve in Mice:

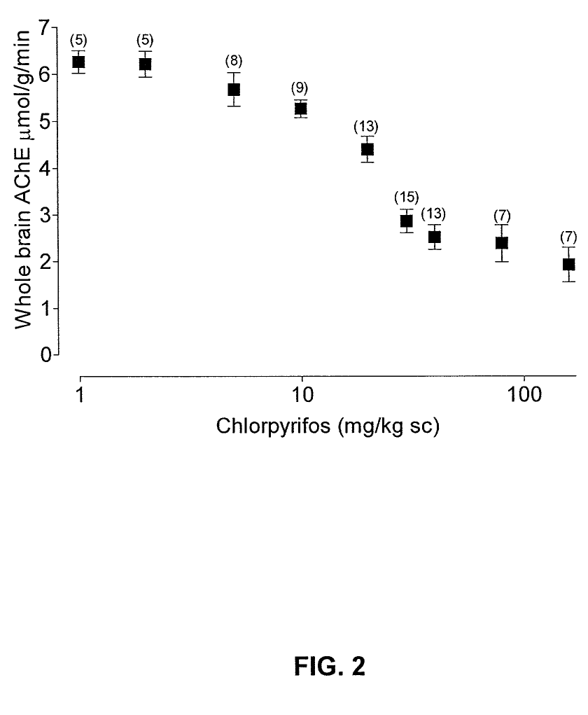

It was necessary to establish a dose-response curve for chlorpyrifos in mice to determine the proper challenge dose. Ten doses of chlorpyrifos (dissolved in DMSO), ranging from 0 to 160 mg/kg, were injected subcutaneously into groups of 5 to 18 mice. 18-24 hours later whole brain AChE was measured. The DMSO alone dose was tested on 18 mice and the average AChE obtained was 5.51±0.174 μmol/g brain/min. The dose-response curve obtained is shown in FIG. 2.

Three of the doses tested were on the linear portion of the curve. The middle of these doses, 30 mg/kg, was chosen as the challenge dose for future experiments. Five of the animals that received this dose had been injected with 200 μl sterile saline four days prior to receiving chlorpyrifos. The average whole brain AChE measured for this dose was 2.864±0.250 μmol/g/min.

Administration of Recombinant Adenoviruses Followed by Chlorpyrifos Challenge:

Three groups of five mice each were injected with 2×10⁹ pfu of either Ad.RR5, Ad.CMV-hPON1-LR or Ad.CMV-hPON1-LQ on Day 0. On Day 3 (45 mice) or Day 5 (5 mice), 100 μl blood was taken from tail veins to assay serum arylesterase and paraoxonase activities. The enzyme activities on these two days differed by 14.9%±4.24% (n=5). On Day 4, each mouse received a subcutaneous injection of 30 mg/kg chlorpyrifos. 18-24 hours later, whole brains were removed and ACHE was measured. This experiment was repeated three times and the data pooled for analysis. Serum enzymes levels are shown in FIGS. 3A & 3B.

Following treatment with the respective viral vectors, serum arylesterase activity was higher in the Ad.CMV-hPON1-LQ-treated group (170.4±8.9 U/ml) and in the Ad.CMV-hPON1-LR-treated group (182.9±12.4) than in the Ad.RR5-virus control group (108.2±7.5, p<0.001), but the levels in the Ad.CMV-hPON1-LQ- and Ad.CMV-hPON1-LR-treated groups were not significantly different (p=0.3, FIG. 3A). The serum paraoxonase activity in the Ad.CMV-hPON1-LR-treated group (393.4±71.3 U/ml) was higher than that in the virus control group (242.5±8.7, p=0.05) and the Ad.CMV-hPON1-LQ-treated group (212.6±14.6, p=0.03), but that in Ad.CMV-hPON1-LQ-treated group was not significantly different from that in the viral control group (p=0.9, FIG. 3B).

Whole brain AChE was measured in mice that received each gene therapy virus or the control virus (Ad.RR5) followed by chlorpyrifos and in two additional control groups, one receiving saline with no virus followed by no chlorpyrifos and another receiving the control virus (Ad.RR5) followed by no chlorpyrifos (FIG. 4). Compared with the large decrease in brain AChE in the group of mice injected with the control virus (Ad.RR5) and later with chlorpyrifos, the groups of mice injected with Ad.CMV-hPON1-LQ and Ad.CMV-hPON1-LR were protected from brain ACHE inhibition by chlorpyrifos (p=0.05 and p=0.001, respectively, FIG. 4). The heterogeneity in the degree of protection (FIG. 4) is a well known feature of gene therapy with the adenoviral vector and should become more uniform as the technique is extended with additional viral vectors.

The Ad.CMV-hPON1-LR virus provided a greater degree of protection than the Ad.CMV-hPON1-LQ virus (p=0.04). Five or six of the animals in the former group were completely protected, as evidenced by their brain ACHE levels remaining the same as the two control groups that received no chlorpyrifos (FIG. 4). The greater protection afforded by the Ad.CMV-hPON1-LR virus was expected because the PON1-LR isoenzyme hydrolyzes chlorpyrifos at least 30% faster than the PON1-LQ isoenzyme (Davies et al. 1996). Since the PON1-LQ isoenzyme more rapidly hydrolyzes other substrates, such as sarin and soman nerve agents (Davies et al., 1996), however, gene therapy to boost the PON1-LQ isoenzyme will provide greater protection from these chemical nerve agents than the PON1-LR isoenzyme. Likewise, the PON1-LQ isoenzyme may be more important in protecting from accelerated atherosclerosis than the PON1-LR isoenzyme (Pfohl et al., 1999; Odawara et al., 1997). Therefore, gene therapy with both isoenzymes will have important uses.

All of the COMPOSITIONS and/or METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and/or METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference:

EPO Patent No. 0273085

U.S. Pat. No. 5,252,479

Abou-Donia et al., Increased neurotoxicity following concurrent exposure to pyridostigmine bromide, DEET, and chlorpyrifos. Fund. Appl. Toxicol. 1996;34:201-22.

Adkins et al., Molecular basis for the polymorphic forms of human serum paraoxonase/arylesterase: glutamine or arginine at position 191, for the respective A or B allozymes. Am. J Hum. Genet. 1993;52:598-608.

Aldridge W N, An enzyme hydrolyzing diethyl p-nitrophenol phosphate (E600) and its identity with the A-esterase of mammalian sera. Biochem. J. 1953;53:117-24.

Ausubel et al., In: Current Protocols in Molecular Biology, John, Wiley & Sons, Inc., 1994.

Baichwal and Sugden, In: Gene Transfer, Kucherlapati R, ed., New York, Plenum Press, pp. 117-148, 1986.

Benvenisty and Neshif, Proc. Natl. Acad. Sci. USA 83(24):9551-9555, 1986.

Betarbet et al., Chronic systemic pesticide exposure reproduces features of Parkinson's disease. Nature Neuroscience 2000;3:1301-6.

Bharucha et al., Geographic distribution of motor neuron disease and correlation with possible etiologic factors. Neurology 1983;33:911-5.

Bog-Hansen et al., Plasma lipoprotein-associated arylesterase is induced by bacterial lipopolysaccharide. FEBS Letters 1978;93 :86-90.

Broomfield et al., Protection by butyrylcholinesterase against organophosphorus poisoning in nonhuman primates. J. Pharm. Exper. Ther. 1991;259:633-8.

Cao et al., Paraoxonase protection of LDL against peroxidation is independent of its esterase activity towards paraoxon and is unaffected by the Q→R genetic polymorphism. J. Lipid Res. 1999;40:133-9.

Caroscio et al., Amyotrophic lateral sclerosis: its natural history. Neurol. Clin. 1987;5:1-8.

Chang et al., 14:124A, 1991.

Checkoway et al., Genetic polymorphisms in Parkinson's disease. Neurotoxicology 1998;19:635-43.

Clendenning et al., Structural organization of the human PON1 gene. Genomics 1996;35:586-9.

Coffin, Retroviridae and Their Replication. In: Virology, Fields et al., eds., Raven Press, New York, pp. 1437-1500, 1990.

Costa et al., Serum paraoxonase and its influence on paraoxon and chlorpyrifos-oxon toxicity in rats. Toxicol. Appl. Pharmacol. 1990; 103:66-76.

Costa et al., The role of paraoxonase (PON1) in the detoxication of organophosphates and its human polymorphism. Chem. Biol. Interact. 1999;119-120:429-38.

Costa L G & Manzo L. Biochemical markers of neurotoxicity: research strategies and epidemiological applications. Toxicology Letters 1995;77: 137-44.

Couch et al., Am. Rev. Resp. Dis., 88:394-403, 1963.

Coulthard et al., Adenovirus-mediated transfer of a gene encoding acyloxyacyl hydrolase (AOAH) into mice increases tissue and plasma AOAH activity. Infect. Immun. 1996;64:1510-5.

Coupar et al., Gene, 68:1-10, 1988.

Davies et al., The effect of the human serum paraoxonase polymorphism is reversed with diazoxon, soman and sarin. Nat. Genet. 1996;14:334-6.

Doctor et al., Cholinesterases as scavengers for organophosphorus compounds: protection of primate performance against soman toxicity. Chem. Biol Interact. 1993;87:285-93.

Doctor et al., Enzymes as pretreatment drugs for organophosphate toxicity. Neuroscience and Biobehavioral Reviews 1991; 15:123-8.

Dubensky et al., Proc. Nat'l Acad. Sci. USA, 81:7529-7533, 1984.

Dunn M A & Sidell F R, Progress in medical defense against nerve agents. JAMA 1989;262:649-52.

Eckerson et al., The human serum paraoxonase polymorphism: identification of phenotypes by their response to salts. Am. J Hum. Genet. 1983;35:214-27.

Eckerson et al., The human serum paraoxonase/arylesterase polymorphism. Am. J. Hum. Genet. 1983;35:1126-38.

Ellman et al., A new and rapid colorimetric determination of acetylcholinesterase activity. Biochem. Pharmacol. 1961;7:88-95.

Fechheimer et al., Proc. Nat'l Acad. Sci USA, 84:8463-8467, 1987.

Feingold et al., Paraoxonase activity in the serum and hepatic mRNA levels decrease during the acute phase response. Atherosclerosis 1998; 139:307-15.

Ferkol et al., FASEB J., 7:1081-1091, 1993.

Friedmann, “Progress toward human gene therapy”, Science, 244:1275-1281, 1989.

Gan et al., Purification of human serum paraoxonase/arylesterase. Drug Metab. Dispos. 1991;19:100-6.

Gerard R D & Meidell R S, Adenoviral Vectors. In: Glover D M, Hames B D, editors. DNA Cloning: A Practical Approach. Vol. 4. Oxford and New York: Oxford University Press; 1995. p. 285-306.

Ghosh and Bachhawat, Targeting of Liposomes to Hepatocytes. In: Liver Diseases, Targeted Diagnosis and Therapy Using Specific Receptors and Ligands. Wu et al., eds., Marcel Dekker, New York, pp. 87-104, 1991.

Gomez-Foix et al., J. Biol. Chem., 267:25129-25134, 1992.

Gopal, Mol. Cell Biol, 5:1188-1190, 1985.

Graham and Prevec, In: Methods in Molecular Biology: Gene Transfer and Expression Protocol, E. J. Murray, ed., Humana Press, Clifton, N.J., 7:109-128, 1991.

Graham, et. al., J. Gen. Virl., 36(1):59-74, 1977.

Gray B, Design and structure-activity relationships of antidotes to p organophosphorous anticholineserase agents. Drug Metabolism Reviews 1984; 15:557-89.

Green M W, Human adenoviruses: growth, purification, and transfection assay. Meth. Enzymol. 1979;58:425-35.

Grunhaus and Horwitz, Seminar in Virology, 3:237-252, 1992.

Haley et al., Association of low PON1 type Q (type A) arylesterase activity with neurologic symptom complexes in Gulf War veterans. Toxicol. Appl. Pharm. 1999;157:227-33.

Haley R W & Kurt T L, Self-reported exposure to neurotoxic chemical combinations in the Gulf War: a cross-sectional epidemiologic study. JAMA 1997;277:231-7.

Haley R W, Gulf War Syndrome: Stress or Neurotoxic Damage? Internal Medicine Grand Rounds and Parkland Memorial Hospital. 10-2-1999 (1999b).

Harland and Weintraub, J. Cell Biol., 101:1094-1099, 1985.

Hassett et al., Characterization of cDNA clones encoding rabbit and human serum paraoxonase: the mature protein retains its signal sequence. Biochemistry 1991;30:10141-9.

Hermonat and Muzycska, Proc. Nat. Acad. Sci USA, 81:6466-6470, 1984.

Hersdorffer et al., DNA Cell Biol., 9:713-723, 1990.

Herz and Gerard, Proc. Natl Acad. Sci. USA, 90:2812-2816, 1993.

Himuro et al., Distal sensory axonopathy after sarin intoxication. Neurology 1998;51:1195-7.

Horwich, et al., J. Virol., 64:642-650, 1990.

Humbert et al., The molecular basis of the human serum paraoxonase activity polymorphism. Nature Genet. 1993;3:73-6.

Husain et al., A comparative study of delayed neurotoxicity in hens following repeated administration of organophosphorus compounds. Indian J. Physiol. Pharmacol. 1995;39:47-50.

Husain et al., Delayed neurotoxic effect of sarin in mice after repeated inhalation exposure. J. Appl. Toxicol. 1993;13:143-5.

Ikeda et al., Serum paraoxonase activity and its relationship to diabetic complications in patients with non-insulin-dependent diabetes mellitus. Metabolism 1998;47:598-602.

Jiang et al., J. Immunol. 154(2):825-31, 1995

Jones and Shenk, Cell, 13:181-188, 1978.

Kaneda et al, Science, 243:375-378, 1989.

Kang et al., Unique cluster of symptoms among Gulf War veterans: factor analysis. Conference of Federally Sponsored Research on Gulf War Veterans' Illnesses Research, Washington, D.C., Jun. 23-35, 1999.

Kao et al., A variant of paraoxonase (PON1) gene is associated with diabetic retinopathy in IDDM. J. Clin. Endocrinol. Metab. 1998;83:2589-92.

Karlsson et al., EMBO J., 5:2377-2385, 1986.

Kato et al., J. Biol. Chem., 266:3361-3364, 1991.

Keeler et al., Pyridostigmine used as a nerve agent pretreatment under wartime conditions. JAMA 1991;266:693-5.

Konda & Yamamoto, Genetic polymorphism of paraoxonase 1 (PON1) and susceptibility to Parkinson's disease. Brain Research 1998;806:271-3.

Ku & Mortensen, Cytokine, 5(4):327-32:1993

La Du B N, Human serum paraoxonase/arylesterase. In: Kalow W, editor. Pharmacogenetics of Drug Metabolism. New York: Pergamon Press, Inc.; 1992. p. 51-91.

La Du et al., Serum paraoxonase (PON1) isozymes: the quantitative analysis of isozymes affecting individual sensitivity to environmental chemicals. Drug Metab. Disposit. 2001 (in press).

Langston J W, Epidemiology versus genetics in Parkinson's disease: progress in resolving an age-old debate. Ann. Neurol. 1998;44 (3 Suppl 1):S45-52.

Le Couteur et al., Pesticides and Parkinson's disease. Biomed. Pharmacother. 1999;53:122-30.

Le Gal La Salle et al., Science, 259:988-990, 1993.

Levrero et al., Gene, 101:195-202, 1991.

Li et al., Serum paraoxonase status: a major factor in determining resistance to organophosphates. J. Toxicol. Environ. Health 1993;40:337-46.

Li et al.; Muscarinic receptor-mediated pyridostigmine-induced neuronal apoptosis Neurotoxicolocy 2000;21:541-52.

Li et al.; Paraopxonase protects against chlorpyrifos toxicity in mice. Toxicology Letters 1995;76-:219-26.

Liao W & Floren C H, Hyperlipidemic response to endotoxin—a part of the host defense mechanism. Scand. J. Infect. Dis. 1993;25:675-82.

Loewenstein-Lichtenstein et al., Genetic predisposition to adverse consequences of anti-cholinesterases in ‘atypical’ BChE carriers. Nature Med. 1995;1:1082-5.

Lorentz et al., Arylesterase in serum: elaboration and clinical application of a fixed-incubation method. Clin. Chem. 1979;25:1714-20.

Lorentz et al., Arylesterase in serum: elaboration and clinical application of a fixed-incubation method. Clin. Chem. 1979;25:1714-20.

Mackness et al., Effect of the molecular polymorphisms of human paraoxonase (PON1) on the rate of hydrolysis of paraoxon. Br. J. Pharmacol. 1997;122:265-8.

Mackness et al., Human serum paraoxonase. Gen. Pharmacol. 1998;31(3):329-36.

Mackness et al., Protection of low-density lipoprotein against oxidative modification by high-density lipoprotein associated paraoxonase. Atherosclerosis 1993; 104:129-35.

Mackness et al., Serum paraoxonase (PON1) 55 and 192 polymorphism and paraoxonase activity and concentration in non-insulin dependent diabetes mellitus. Atherosclerosis 1998;139:341-9.

Mackness et al., The separation of sheep and human serum A-esterase activity into the lipoprotein fraction by ultracentrifugation. Comp. Biochem. Physiol. 1985;82B:675-7.

Magari et al., Pharmacologic control of a humanized gene therapy system implanted into nude mice. J. Clin. Invest. 1997;100:2865-72.

Mann et al., Cell, 33:153-159, 1983.

Markowitz et al., J. Virol., 62:1120-1124, 1988.

McGeer et al., Familial nature and continuing morbidity of the amyotrophic lateral sclerosis-parkinsonism dementia complex of Guam. Neurology 1997;49:400-9.

McGuire et al., Occupational exposures and amyotrophic lateral sclerosis: a population-based case-control study. Am. J. Epidemiol. 1997;145:1076-88.

Morsy et al., An adenoviral vector deleted for all viral coding sequences results in enhanced safety and extended expression of a leptin transgene. Proc. Nat'l Acad. Sci USA 1998;95:7866-71.

Mulligan, Science, 260:926-932, 1993.

Murata et al., Asymptomatic sequelae to acute sarin poisoning in the central and autonomic nervous system 6 months after the Tokyo subway attack. J. Neurol. 1997;244:601-6.

Nicolas and Rubinstein, In: Vectors: A survey of molecular cloning vectors and their uses, Rodriguez and Denhardt, eds., Stoneham: Butterworth, pp. 494-513, 1988.

Nicolau et al., Methods Enzymol., 149:157-176, 1987.

Odawara et al., Paraoxonase polymorphism (Gln192-Arg) is associated with coronary heart disease in Japanese noninsulin-dependent diabetes mellitus. J. Clin. Endocrinol. Metab. 1997;82:2257-60.

Oyanagi & Wada, Neuropathology of parkinsonism-dementia complex and amyotrophic lateral sclerosis of Guam: an update. J. Neurol. 1999;246 (Suppl 2):II19-27.

Paskind et al., Virology, 67:242-248, 1975.

Perales et al., Proc. Nat'l Acad. Sci. 91:4086-4090, 1994.

Pfohl et al., Paraoxonase 192 Gln/Arg gene polymorphism, coronary artery disease, and myocardial infarction in type 2 diabetes. Diabetes 1999;48:623-7.

Playfer et al., Genetic polymorphism and interethnic variability of plamsa paraoxonase activity. J. Med. Genet. 1976;13:337-42.

Poirier et al., Environment, genetics and idiopathic Parkinson's disease. Can. J. Neurol. Sci. 1991;18:70-6.

Potter et. al., Proc. Nat'l Acad. Sci. USA, 81:7161-7165, 1984.

Primo-Parmo et al., The human serum paraoxonase/arylesterase gene (PON1) is one member of a multigene family. Genomics 1996;33:498-507.

Racher et al., Biotechnology Techniques, 9:169-174, 1995.

Ragot et al., Nature, 361:647-650, 1993.

Renan, Radiother. Oncol., 19:197-218, 1990.

Rich et al., Hum. Gene Ther., 4:461-476, 1993.

Ridgeway, Mammalian Expression Vectors, In: Vectors: A Survey of Molecular Cloning Vectors and Their Uses, Rodriguez et al., eds., Stoneham: Butterworth, pp. 467-492, 1988.

Rivera et al., A humanized system for pharmacologic control of gene expression. Nature Med. 1996;2: 1028-32.

Rosenfeld et al., Cell, 68:143-155, 1992.

Rosenfeld et al., Science, 252:431-434, 1991.

Roux et al., Proc. Nat'l Acad Sci. USA, 86:9079-9083, 1989.

Sakai et al., Serum paraoxonase activity and genotype distribution in Japanese patients with diabetes mellitus. Intern. Med. 1998;37:581-4.

Sambrook et. al., In: Molecular Cloning: A Laboratory Manual, 2d Ed., Cold Spring Harbor Laboratory Press, Cold Spring Harbor, N.Y., 1989.

Samulski et al., J. Virol., 61(10):3096-3101, 1987.

Shih et al., Mice lacking serum paraoxonase are susceptible to organophosphate toxicity and atherosclerosis. Nature 1998;394:284-7.

Sidell F R, Soman and sarin: clinical manifestations and treatment of accidental poisoning by organophosphates. Clin. Toxicol. 1974;7:1-17.

Sinton et al., Delayed, prolonged behavioral effects of treatment with low doses of pyridostigmine in the rat. Under journal peer review 2001.

Smith et al., Killing of trypanosomes by the human haptoglobin-related protein. Science 1995;268:284-6.

Sorenson et al., Reconsideration of the catalytic center and mechanism of mammalian paraoxonase/arylesterase. Proc. Nat'l Acad. Sci. USA 1995b;92:7187-91.

Sorenson et al., The genetic mapping and gene structure of mouse paraoxonase/arylesterase. Genomics 1995a;30:431-8.

Stratford-Perricaudet and Perricaudet, In: Human Gene Transfer, O. Cohen-Haguenauer et al., eds., John Libbey Eurotext, France, pp. 51-61, 1991.

Stratford-Perricaudet et al, Hum. Gene. Ther., 1:241-256, 1990.

Temin, In: Gene Transfer, Kucherlapati R, ed., New York, Plenum Press, pp. 149-188, 1986.

Top et. al., J. Infect. Dis., 124:155-160, 1971.

Tucker J B, Evidence Iraq used chemical weapons during the 1991 Persian Gulf War. The Nonproliferation Review 1997;Spring-Summer:114-22.

Tur-Kaspa et al., Mol. Cell Biol., 6:716-718, 1986.

U.S. Senate Committee Report on Banking, Housing and Urban Affairs, United States Senate. U.S. chemical and biological warfare-related dual use exports to Iraq and their possible impact on the health consequences of the Persian Gulf War. Washington: U.S. Senate, 1994.

Varmus et al., Cell, 25:23-36, 1981.

Wagner et al., Proc. Nat'l Acad. Sci. USA 87(9):3410-3414, 1990.

Wong et al., Gene, 10:87-94, 1980.

Wu and Wu, Adv. Drug Delivery Rev., 12:159-167, 1993.

Wu and Wu, Biochemistry, 27:887-892, 1988.

Wu and Wu, J. Biol. Chem., 262:4429-4432, 1987.

Yang et al., Cellular immunity to viral antigens limits E1-deleted adenoviruses for gene therapy. Proc. Nat'l Acad. Sci. USA 1994;91:4407-11.

Ye et al., Regulated delivery of therapeutic proteins after in vivo somatic cell gene transfer. Science 1999;283:88-91.

Yokoyama et al., A preliminary study of delayed vestibulocerebellar effects of Tokyo subway sarin poisoning in relation to gender difference: frequency analysis of postural sway. J. Occu.p Environ. Med. 1998;40: 17-21.

Yokoyama et al., Chronic neurobehavioral and central and autonomic nervous system effects of Tokyo subway sarin poisoning. J. Physiol. Paris 1998;92:317-23.

Zaki et al., Potential toxins of acute liver failure and their effects on blood-brain barrier permeability. Experientia 1983;39:988-91.

What is claimed is:

1. A method of protecting a cell from a toxin comprising:
(a) providing an expression cassette comprising a promoter active in said host cell and a gene encoding PON1 under the control of said promoter; and
(b) transferring said expression cassette into said cell under conditions permitting expression of PON1.

2. The method of claim 1, wherein PON1 is PON1 type Q.

3. The method of claim 1, wherein PON1 is PON1 type R.

4. The method of claim 1, wherein said cell expresses PON1 type Q.

5. The method of claim 1, wherein said cell expresses PON1 type R.

6. The method of claim 1, wherein said toxin is an organophosphate.

7. The method of claim 6, wherein said organophosphate is an organophosphate pesticide.

8. The method of claim 1, wherein said toxin is a nerve agent.

9. The method of claim 1, wherein said expression cassette further comprises a polyadenylation signal.

10. The method of claim 1, wherein said expression cassette is further comprised within a vector.

11. The method of claim 10, wherein said vector is a viral vector.

12. The method of claim 11, wherein said viral vector is a herpesviral vector, a retroviral vector, an adenoviral vector, an adeno-associated viral vector, a polyoma viral vector, and a vaccinia viral vector.

13. The method of claim 11, wherein said viral vector is an adenoviral vector.

14. The method of claim 1, wherein said promoter is a constitutive promoter.

15. The method of claim 1, wherein said promoter is an inducible promoter.

16. The method of claim 1, wherein said promoter is a tissue specific promoter.

17. The method of claim 4, wherein said expression cassette increases PON1 type Q expression by about 10-fold.

18. The method of claim 5, wherein said expression cassette increases PON1 type R expression by about 10-fold.

19. The method of claim 1, wherein said cell is a liver cell.

20. The method of claim 1, wherein said cell expresses low levels of PON1 type Q or R as compared to the general population.

21. A method of protecting a subject from a toxin comprising:
(a) providing an expression cassette comprising
(i) a promoter active in host cells of said subject,
(ii) a gene encoding PON1 under the control of said promoter; and
(b) administering to said subject said expression cassette under conditions permitting expression of PON1.

22. The method of claim 21, wherein said toxin is an organophosphate.

23. The method of claim 22, wherein said organophosphate is an organophosphate pesticide.

24. The method of claim 21, wherein said toxin is a nerve agent.

25. The method of claim 21, wherein administering comprises intravenously or intraarterially.

26. A method for protecting a subject from chemical warfare agents comprising:
(a) determining a chemical warfare threat;
(b) providing to said subject an expression cassette comprising
(i) a promoter active in host cells of said subject,
(ii) and a gene encoding PON1 under the control of said promoter, in a form suitable for self administration; and
(c) providing to said subject information of said chemical warfare threat and instructions on the self administration of said expression cassette.

27. The method of claim 26, wherein said form suitable for self administration is a pharmaceutical preparation of a virus particle comprising said expression cassette.

28. The method of claim 26, wherein said PON1 is PON1 type Q.

29. A method of protecting a subject from chemical warfare agents comprising administering to said subject an expression cassette comprising:
(a) a promoter active in cells of said subject; and
(b) a gene encoding PON1 under the control of said promoter under conditions permitting expression of PON1.

30. The method of claim 29, wherein said form suitable for self administration is a pharmaceutical preparation of a infectious virus comprising said expression cassette.

31. The method of claim 29, wherein said PON1 is PON1 type Q.

32. A method of treating a subject to protect, correct or retard the progress of a neurodegenerative disease comprising administering to said subject an expression cassette comprising:
(a) a promoter active in cells of said subject; and
(b) a gene encoding PON1 under the control of said promoter under conditions permitting expression of PON1.

33. The method of claim 32, wherein said neurodegenerative disease is Parkinson's Disease or amyotropic lateral sclerosis.

34. A method of treating or protecting a subject from atherosclerosis comprising administering to said subject an expression cassette comprising:
(a) a promoter active in cells of said subject; and
(b) a gene encoding PON1 under the control of said promoter under conditions permitting expression of PON1.

35. A method of treating or protecting a subject from Gulf War Syndrome comprising administering to said subject an expression cassette comprising:
(a) a promoter active in cells of said subject; and
(b) a gene encoding PON1 under the control of said promoter under conditions permitting expression of PON1.
